(19) United States
(12) Patent Application Publication
(10) Pub. No.: US 2015/0224224 A1
(43) Pub. Date: Aug. 13, 2015

(54) BIOACTIVATED MATERIAL

(71) Applicant: NOBEL BIOCARE SERVICES AG, Kloten (CH)

(72) Inventor: Günter Uhr, Zurich (CH)

(21) Appl. No.: 14/596,102

(22) Filed: Jan. 13, 2015

Related U.S. Application Data
(63) Continuation-in-part of application No. PCT/EP2013/001985, filed on Jul. 5, 2013.
Application No. PCT/EP2013/001985 is a continuation-in-part of application No. PCT/EP2013/001981, filed on Jul. 5, 2013.

(30) Foreign Application Priority Data
Jul. 14, 2012 (GB) 1212568.8
Jul. 14, 2012 (GB) 1212569.6

(51) Int. Cl. A61L 27/32; A61L 27/28; A61L 27/54

(52) U.S. Cl. CPC A61L 27/32; A61L 27/54; A61L 27/28; A61L 2430/02; A61L 2420/06; A61L 2300/412; A61L 2300/21; A61L 2300/232; A61L 2300/112; A61L 2300/204

(57) ABSTRACT

The present invention generally relates to the field of bone substitute materials, and particularly to a bone substitute material that is bioactivated. More specifically, the present invention relates to a particular agent comprising citrate, citric acid, monobasic sodium phosphate, dextrose, and adenine for use in improving or promoting osseointegration of a bone substitute material, to a bone substitute material provided with a coating of said particular agent, and to a kit for use in a method of installing a bone substitute material comprising a bone substitute material as well as citrate, citric acid, monobasic sodium phosphate, dextrose, and adenine.

### CROSS-REFERENCE TO RELATED APPLICATIONS

This application is a continuation-in-part of International Application No. PCT/EP2013/001985, filed on Jul. 5, 2013, which claims priority benefit of GB Patent Application No. 1212569.6, filed on Jul. 14, 2012. This application also is a continuation-in-part of International Application No. PCT/EP2013/001981, filed on Jul. 5, 2013, which claims priority benefit of GB Patent Application No. 1212568.8, filed on Jul. 14, 2012. Each of the foregoing applications is expressly incorporated by reference in its entirety.

### FIELD OF THE INVENTION

The present invention generally relates to the field of bone substitute materials, and particularly to a bone substitute material that is bioactivated. More specifically, the present invention relates to a particular agent comprising citrate, citric acid, monobasic sodium phosphate, dextrose, and adenine for use in improving or promoting osseointegration of a bone substitute material, to a bone substitute material provided with a coating of said particular agent, and to a kit for use in a method of installing a bone substitute material comprising a bone substitute material as well as citrate, citric acid, monobasic sodium phosphate, dextrose, and adenine.

### BACKGROUND OF THE INVENTION

Bone substitute materials are used to supplement or substitute autogenous cancellous bone. In comparison to the transplantation of cancellous bone, the bone substitutes have the advantages that they are sufficiently available and can be stored. Bone substitute materials serve as filling materials to mechanically strengthen the defect site by providing a scaffold structure and to support bone healing.

Bone substitute materials may be classified by their biological mechanisms: osteoconduction, osteoinduction and osteogenesis.

Osteoconduction occurs when the bone substitute material serves as a scaffold for new bone growth that is perpetuated by the native bone. Osteoblasts from the margin of the defect site utilize the bone substitute materials as a framework upon which new bone is generated. In the very least, a bone substitute material should be osteoconductive. Typical osteoconductive materials include synthetic materials such as ceramics of calcium phosphates (e.g. hydroxyapatite and tricalcium phosphate), biopolymers, bioglass and calcium phosphate, as well as allogenic and xenogenic materials, such as bovine.

Osteoinduction involves the stimulation of osteoprogenitor cells to differentiate into osteoblasts that then begin new bone formation. The most widely studied type of osteoinductive cell mediators are bone morphogenetic proteins (BMP's). A bone substitute material that is osteoconductive and osteoinductive will not only serve as a scaffold for currently existing osteoblasts but will also trigger the formation of new osteoblasts. Besides BMP's osteoinductive materials include demineralized bone matrix and autogenous bone grafts of milled bone.

Osteogenesis occurs when vital osteoblasts originating from the bone substitute material contribute to new bone growth along with bone growth generated via the other two mechanisms. Materials that show osteogenesis include autogenous bone and tissue engineered materials.

Bone substitute materials usually have a porous structure and are available in granular, block, and putty form, either soft and pliable, or hard and form keeping. All bone substitute materials require blood penetration such that the fibrin network can be formed during healing. A resorbable bone substitute materials should degrade or resorb simultaneously while bone is formed. Non-resorbable bone substitute materials should be osseointegrated. A prerequisite of osseointegration is a direct structural and functional connection between the living bone and the bone substitute material without the interposition of connective tissue.

It has been suggested that wettability of a bone substitute material has a high impact on osseointegration. For example, when a hydrophobic bone substitute material, such a polycaprolactone-based polyurethane urea, is exposed to blood, it shows no penetration of the fibrin within the porous structure. On the other hand, hydrophilic bone substitute materials may induce accelerated platelet activation at the material's periphery but the clotted blood may form a capsule around the substitute material inhibiting complete blood penetration. Therefore, although good wettability is an important factor, there are also others, still unknown properties that add to a good osseointegration.

In view of the above the object to be solved by the present invention is the provision of an agent having the ability of improving osseointegration of a bone substitute material as well as the provision of a bone substitute material having excellent osseointegration properties.

### SUMMARY OF THE INVENTION

The present inventor has surprisingly found that the presence of citrate, phosphate, dextrose and adenine on the surface of a bone substitute material induces blood protein coating thereon, platelet activation and a tight fibrin fiber connection to such a surface. These effects, i.e. an improved blood protein coating, an improved platelet activation and a tight fibrin fiber connection, are basic requirements for an excellent osseointegration of a bone substitute material.

In view of the above finding, in a first aspect, the present invention relates to an agent for use in improving or promoting osseointegration of a bone substitute material, wherein the agent comprises trisodium citrate, citric acid, monobasic sodium phosphate, dextrose, and adenine.

In a second aspect, the present invention relates to the use of an agent for producing a coating on a bone substitute material, wherein the agent comprises trisodium citrate, citric acid, monobasic sodium phosphate, dextrose, and adenine.

In a third aspect, the present invention relates to a bone substitute material characterized in that the bone substitute material is provided with at least one coating, wherein the coating is obtainable by immersing the bone substitute material in an aqueous solution comprising trisodium citrate, citric acid, monobasic sodium phosphate, dextrose, and adenine.

In a fourth aspect, the present invention relates to a kit for use in a method of installing a bone substitute material wherein the kit comprises a bone substitute material, trisodium citrate, citric acid, monobasic sodium phosphate, dextrose and adenine.

### BRIEF DESCRIPTION OF THE DRAWINGS

FIG. 1a shows a SEM photograph (magnification=1,000-fold) of an Artelon® membrane after exposure to uncoagulated blood, wherein the Artelon® membrane has not been provided with a coating as described in the present application.

FIG. 1b shows a SEM photograph (magnification=1,000-fold) of an Artelon® membrane according to the present invention after exposure to uncoagulated blood.

FIG. 2a shows a SEM photograph (magnification=14,000-fold) of a bone substitute material of the type Cerasorb Gelatine after exposure to uncoagulated blood, wherein the bone substitute membrane has not been provided with a coating as described in the present application.

FIG. 2b shows a SEM photograph (magnification=14,000-fold) of a bone substitute material of the type Cerasorb Gelatine according to the present invention after exposure to uncoagulated blood.

### DETAILED DESCRIPTION

According to the first aspect, the present invention provides an agent for use in a method of improving or promoting osseointegration of a bone substitute material, wherein the agent comprises trisodium citrate, citric acid, monobasic sodium phosphate, dextrose and adenine.

According to a preferred embodiment of the present invention the agent is dissolved in water forming an aqueous solution. It is particularly preferred that this aqueous solution for use in improving or promoting osseointegration of a bone substitute material comprises:

1.32 to 105.20 g/l, preferably 2.63 to 26.3 g/l of trisodium citrate,

0.16 to 13.08 g/l, preferably 0.33 to 3.27 g/l of citric acid,

0.11 to 8.88 g/l, preferably 0.22 to 2.22 g/l of monobasic sodium phosphate,

1.60 to 127.60 g/l, preferably 3.19 to 31.90 g/l of dextrose, and

0.01 to 1.1 g/l, preferably 0.03 to 0.28 of adenine.

Such an aqueous solution (in the following also referred to as “the coating solution”) as defined above is preferred because an aqueous solution containing 26.3 g/l of trisodium citrate, 3.27 g/l of citric acid, 2.22 g/l monobasic sodium phosphate, 31.9 g/l dextrose and 0.275 g/l adenine is a solution already approved by the FDA and the European authorities. Such a solution, so-called CPDA-1 solution, is established as an anticoagulant-preservative and adjuvant for blood bags to enable storage for 35 days without substantial loss of blood cell vitality.

According to a particularly preferred embodiment, the solution for use according to the present invention is a 5 to 40 vol.-% solution of the above-mentioned CPDA-1 solution. According to the most preferred embodiment it is used a 10 to 20 vol.-% solution of the above-mentioned CPDA-1 solution.

In other words, according to a particularly preferred embodiment of the present invention it is used an aqueous solution consisting of 2.63 to 5.26 g/l of trisodium citrate, 0.327 to 0.654 g/l of citric acid, 0.222 to 0.444 g/l of monobasic sodium phosphate, 3.19 to 6.38 g/l of dextrose, and 0.0275 to 0.055 g/l of adenine, and the rest is water.

The “bone substitute material” according to the present invention can be any bone substitute material known in the art which would benefit from improved osseointegration. The bone substitute material used according to the present invention may be resorbable or non-respobable. Furthermore, the bone substitute material used according to the present invention may have either osteoconductive properties or osteoconductive plus osteoinductive properties. Typical bone substitute materials to be used in the present invention include synthetic, artifical and natural bone substitute materials. As examples can be mentioned ceramic-based bone substitute materials, such as ceramic-based tricalcium phosphate, a calcium phosphate cement, a bovine-derived hydroxyapatite and polymer-based materials made of e.g. polycaprolactone and/or polyurethane. According to a preferred embodiment of the present invention, the bone substitute material contains tricalcium phosphate. According to an even more preferred embodiment, the bone substitute material is a beta-tricalcium phosphate or a mixture of beta-tricalcium phosphate and porcine gelatine.

It is also possible that the bone substitute material is additionally provided with means of improving or promoting the osteoinductive and/or osteogenic properties of the bone substitute material. It is also possible to use a dental support component having a surface that is provided with growth factors, preferably transforming growth facors Beta, e.g. bone morphogenetic protein (BMP-2, -4, -7), or in combination with platelet rich plasma (PRP). Growth factors may further include any one selected from the group consisting of platelet-derived growth factor (PDGF), vascular endothelial growth factor (VEGF), insulin-like growth factor (IGF), fibroblast growth factors (FGF), differentiation factor 5 (GDF-5).

Osseointegration is the direct structural and functional connection between living bone and the surface of the bone substitute material. An intense blood protein coating and an intense fibrin network integration of the surface of the bone substitute material will influence the cascade of cell-interrogation, recruitment, proliferation, differentiation and maturation of matrix. Fibrin fibers which form the preliminary matrix are “highways” on which soft tissue and hard tissue forming cells move along to the surface of the bone substitute material. Proteins and growth factors released from activated platelets support and organize cellular communication. The platelet-released factors also induce and amplify the migration, proliferation and differentiation of endothelial cells and thereby initiating angiogenesis. The invasion and sprouding of blood vessels is a prerequisite for a sufficient nutrition and oxygen supply, demanded by highly active regenerative cells. By optimizing the wound healing processes on the surfaces of a bone substitute material, the contact-osteogenesis will occur earlier and finally osseointegration will be accelerated.

According to the second aspect of the present invention, the above-defined agent is used for producing a coating on the bone substitute material as defined above. As explained above, according to a preferred embodiment of the present invention, the agent is dissolved in water forming an aqueous solution. Preferably, the aqueous solution for producing a coating on the surface of the bone substitute material is as defined above.

The method of coating the bone substitute material with the above-defined agent is not particularly limited and any known method may be employed.

In case of using the agent in form of an aqueous solution as defined above, the bone substitute material may be immersed in the aqueous solution, i.e. the above-defined aqueous solution, at ambient temperature (25° C.) for e.g. 10 min. In case of a porous bone substitute material it is recommendable to apply a low vacuum (e.g. 600 mbar) for e.g. about 10 min to ensure that the coating solution penetrates into the pores. Subsequently, the bone substitute material is removed from the solution and air-dried by e.g. 37° C. for 30 min. A drying of the coated material is not essential though. Alternatively, it is also possible to directly use the as-coated bone substitute material, i.e. without drying, for implantation.

The above exemplified method can be suitably modified, if desired. For example, it is possible to apply a vacuum to the bone substitute material first, thereby removing water adsorbed on the surface. Such a pre-treatment is preferred in case of a porous bone substitute material because it is beneficial to remove adsorbed water inside the pores before carrying out the coating procedure. Afterwards the so-treated material is coated with the coating solution by e.g. immersing the bone substitute material in the coating solution.

Especially in case of a non-porous bone substitute material where a penetration of the coating solution into pores is not an issue it is possible to pour the coating solution directly into the wound, i.e. defect site, of a patient, immediately before the bone substitute material is inserted in the hard or soft tissue. Thereby, it is possible to obtain a covering of the bone substitute material with the coating solution.

It is also possible to apply the coating of the surface of the bone substitute material step by step using single-component solutions. For example, it is possible to first immerse the bone substitute material in a solution containing trisodium citrate, thereafter in a solution containing citric acid, afterwards the bone substitute material is immersed in a solution containing monobasic sodium phosphate, after this the so-treated material is immersed in a solution containing dextrose and finally the material is immersed in a solution containing adenine. The single-component solutions can be applied to the surface of the bone substitute material in any order. Alternatively, it is also possible to prepare solutions of two, three or four of the above-mentioned agents prior to the coating step.

In case the agent is not dissolved in water, as alternative methods for applying the coating to the bone substitute material chemical vapor deposition and exposure to the dry crystalline agents can be mentioned.

According to the third aspect, the present invention provides a bone substitute material that is provided with at least one coating, wherein said coating is obtainable by immersing the bone substitute material in an aqueous solution comprising trisodium citrate, citric acid, monobasic sodium phosphate, dextrose and adenine. The aqueous solution is the same as specified above.

The thickness of the coating is not specifically limited and may be between a few micrometers to some millimeters. The thickness of the coating depends on the concentration of the coating solution and the coating conditions. In general it can be said that a higher concentrated coating solution increases the thickness of the coating on the bone substitute material. Alternatively, if it is intended to increase the amount of coating on the bone substitute material, it is possible to repeat the coating step as often as desired.

Furthermore, the surface of the bone substitute material coated with the above-defined agent, in particular with the above-defined coating solution, comprising trisodium citrate, citric acid, monobasic sodium phosphate, dextrose and adenine, is preferably hyperhydrophilic. The wettability of the coated bone substitute materials may be determined by measuring the contact angle according to the Sessile-Drop-Method. The coated bone substitute material according to the present invention shows a contact angle of below 30°, in particular of about 20°, at the time t=0s (time of the first contact of the water droplet with the surface). However, the water droplet completely spreads on the surface within some seconds, such as below 50 ms, more particular about 5 to 10 ms. Such a wetting behavior is typical for hydrophilic surfaces.

In a fourth aspect, the present invention relates to a kit for use in a method of installing a bone substitute material, wherein the kit comprises a bone substitute material, trisodium citrate, citric acid, monobasic sodium phosphate, dextrose and adenine.

It is possible that the kit according to the fourth aspect contains trisodium citrate, citric acid, monobasic sodium phosphate, dextrose and adenine as dry agents packaged in the form of dry powders. Such dry powders may be packaged separately or as a mixture of two or more or all of the dry agents. Before coating of the bone substitute material, these dry powders can be dissolved in an appropriate amount of water to prepare the above-defined coating solution.

According to a further preferred embodiment of the fourth aspect of the present invention, the kit contains an aqueous solution containing trisodium citrate, citric acid, monobasic sodium phosphate, dextrose and adenine. It is preferred that the kit contains the aqueous solution according to the above described preferred embodiments. For examples, the aqueous solution to be contained in the kit of the present invention consists of 2.63 to 5.26 g/l of trisodium citrate, 0.327 to 0.654 g/l of citric acid, 0.222 to 0.444 g/l of monobasic sodium phosphate, 3.19 to 6.38 g/l of dextrose, and 0.0275 to 0.055 g/l of adenine, and the rest is water.

### EXAMPLES

### Example 1

It has been used the bone substitute material Artelon® of a membrane-type. Artelon® is a biodegradable polyurethane urea. This material has been coated with an aqueous coating solution having the following composition:

2.63 g/l of trisodium citrate,

0.327 g/l of citric acid,

0.222 g/l monobasic sodium phosphate,

3.19 g/l dextrose and

0.0275 g/l adenine.

The bone substitute material has been immersed in the coating solution for 10 min. Then, low vacuum of 600 mbar has been applied. After 10 min, the bone substitute material has been removed from the coating solution and dried at 37° C. for 20 min.

For determining the coagulation of blood in direct contact with the surface of the bone substitute material the coated material has been exposed to fresh (i.e. uncoagulated) human blood. The coagulation property serves as an indication of beneficial conditions for enabling osseointegration. Therefore, the coated material was placed in an Eppendorf vial and the blood was added. After 10 min the material was removed and fixed in a formaldehyde solution. The so-fixed material was stored over night at 4° C.

For comparative reasons, the same bone substitute material Artelon® of a membrane-type but which has not been provided with the above-described coating has been treated with blood in the same way as described above.

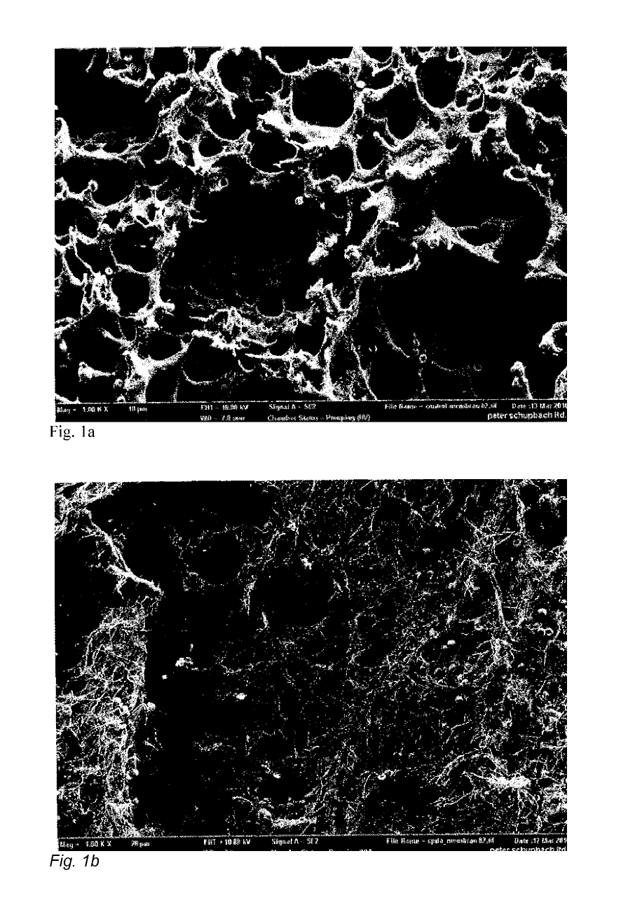

FIG. 1a shows a SEM photograph (magnification=1,000-fold) of the bone substitute material after exposure to uncoagulated blood, wherein the material has not been provided with a coating according to the present invention.

FIG. 1b shows a SEM photograph (magnification=1,000-fold) of the bone substitute material according to the present invention after exposure to uncoagulated blood.

It is apparent from the comparison of FIG. 1a with FIG. 1b that the blood protein coating, the platelet activation and fibrin fiber connection is greatly improved on the surface of the bone substitute material according to the present invention compared to an untreated bone substitute material.

### Example 2

A bone substitute material of the type Cerasorb Gelatine has been coated in the same way as described in Example 1.

The coated bone substitute material has been treated with blood in the same way as described in Example 1.

Furthermore, for comparative reasons, the non-coated bone substitute material has been treated with blood in the same way.

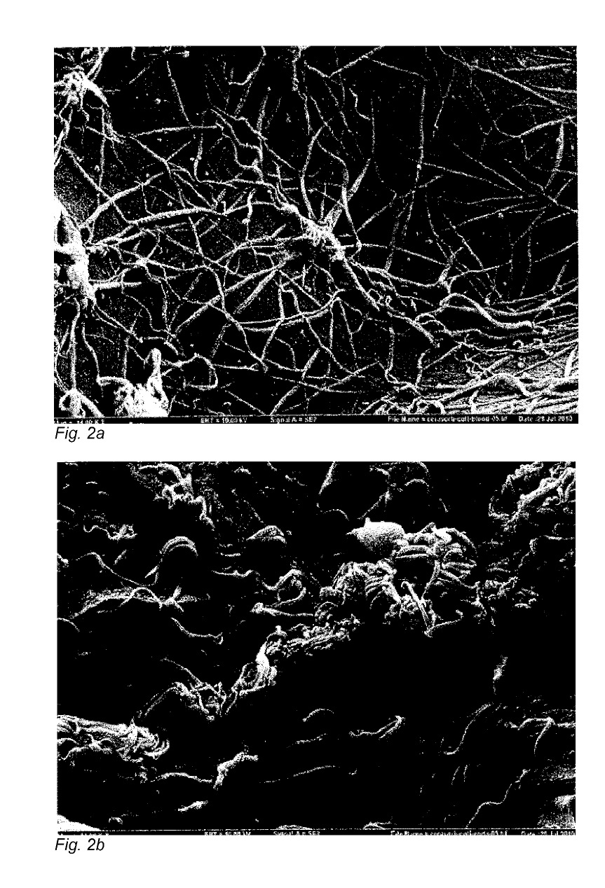

The surfaces of the blood-treated bone substitute materials are shown in FIGS. 2a and 2b. It is apparent from the comparison of FIG. 2a with FIG. 2b that the blood protein coating, the platelet activation and fibrin fiber connection is greatly improved on the surface of the bone substitute material according to the present invention compared to an untreated bone substitute material.

1. An agent for use in improving or promoting osseointegration of a material to be placed in living bone, wherein the agent comprises trisodium citrate, citric acid, monobasic sodium phosphate, dextrose, and adenine.

2. The agent according to claim 1, wherein the material contains tricalcium phosphate.

3. The agent according to claim 1, wherein the agent is dissolved in water forming an aqueous solution.

4. The agent according to claim 3, wherein the aqueous solution comprises
1.32 to 105.20 g/l of trisodium citrate,
0.16 to 13.08 g/l of citric acid,
0.11 to 8.88 g/l of monobasic sodium phosphate,
1.60 to 127.60 g/l of dextrose, and
0.01 to 1.10 g/l of adenine.

5. A method of improving or promoting osseointegration of a material to be placed in living bone, the method comprising:
coating the material with an agent, wherein the agent comprises trisodium citrate, citric acid, monobasic sodium phosphate, dextrose, and adenine.

6. The method according to claim 5, wherein the material contains tricalcium phosphate.

7. The method according to claim 5, wherein coating the material comprises immersing the material with an aqueous solution comprising the agent dissolved in water.

8. The method according to claim 7, wherein the aqueous solution comprises
1.32 to 105.20 g/l of trisodium citrate,
0.16 to 13.08 g/l of citric acid,
0.11 to 8.88 g/l of monobasic sodium phosphate,
1.60 to 127.60 g/l of dextrose, and
0.01 to 1.10 g/l of adenine.

9. A material to be placed in living bone, the material comprising at least one coating, the coating comprising trisodium citrate, citric acid, monobasic sodium phosphate, dextrose, and adenine.

10. The material according to claim 9, wherein the material contains tricalcium phosphate.

11. The material according to claim 9, wherein the coating comprises an aqueous solution comprising:
1.32 to 105.20 g/l of trisodium citrate,
0.16 to 13.08 g/l of citric acid,
0.11 to 8.88 g/l of monobasic sodium phosphate,
1.60 to 127.60 g/l of dextrose, and
0.01 to 1.10 g/l of adenine.

12. The material according to claim 9, wherein the surface of the material is hydrophilic.

13. A kit for use in a method of installing a material in living bone, wherein the kit comprises the material, trisodium citrate, citric acid, monobasic sodium phosphate, dextrose and adenine.

14. The kit according to claim 13, wherein trisodium citrate, citric acid, monobasic sodium phosphate, dextrose and adenine are present in an aqueous solution.

15. The agent according to claim 1, wherein the material is a bone substitute material.

16. The agent according to claim 1, wherein the material is a ceramic or metal of an implant.

17. The method according to claim 5, wherein the material is a bone substitute material.

18. The method according to claim 5, wherein the material is a ceramic or metal of an implant.

19. The material according to claim 9, wherein the material is a bone substitute material.

20. The kit according to claim 13, wherein the material is a bone substitute material.
